(19) United States
(12) Patent Application Publication
(10) Pub. No.: US 2023/0165983 A1
(43) Pub. Date: Jun. 1, 2023

(54) Appliance Disinfecting Illumination

(71) Applicant: Vyv, Inc., Latham, NY (US)

(72) Inventors: Cori Winslow, Rensselaer, NY (US); Robert Barron, Boulder, CO (US); Kasia Kozak, Troy, NY (US); Aram Kuzmak, Troy, NY (US)

(21) Appl. No.: 17/957,289

(22) Filed: Sep. 30, 2022

Related U.S. Application Data
(63) Continuation of application No. 16/728,931, filed on Dec. 27, 2019, now abandoned.
(60) Provisional application No. 62/786,722, filed on Dec. 31, 2018.

(51) Int. Cl. A61L 2/08; A61L 2/24

(52) U.S. Cl. CPC A61L 2/084; A61L 2/24; A61L 2202/11; A61L 2202/14

(57) ABSTRACT

Devices, methods, and systems for disinfection with light are disclosed. In some examples, a lighting source is operable to provide light at wavelength range of about 380 nm - 420 nm with an irradiance and/or dosage sufficient for disinfection. One or more sensors and a control system may be used to control operation of the lighting source, such as by adjusting the lighting source in response to various inputs.

### CROSS-REFERENCE TO RELATED APPLICATIONS

This application is a continuation of U.S. Pat. Application No. 16/728,931, titled “Appliance Disinfecting Illumination” and filed on Dec. 27, 2019, which claims the benefit of U.S. Provisional Application No. 62/786,722, titled “Appliance Disinfecting Illumination” and filed on Dec. 31, 2018. Each of the above-referenced applications is hereby incorporated by reference in its entirety.

### TECHNICAL FIELD

Aspects of the present disclosure generally relate to using illumination for disinfecting appliances.

### BACKGROUND

Many consumer devices may be inhabited by harmful microorganisms such as bacteria, mold, fungi, etc. In some examples, microbial contamination may result from normal usage of an appliance. For example, a food storage appliance may contain bacteria within it. Many kitchen appliances, such as refrigerators, are in contact with raw meat and vegetables that may contain bacteria. Consumption of food products carrying harmful microorganisms or that are in contact with contaminated surfaces may lead to food-borne illnesses. Many microorganisms may create unpleasant odors within consumer devices. As another example, user interaction with an appliance may result in microbial contamination (e.g., on an appliance handle, a door handle). Microorganisms may transfer to other users, through contact of the same consumer devices, and may result in illness. Harmful bacteria such as Escherichia coli (E. coli), Salmonella, Methicillin-resistant Staphylococcus aureus (MRSA), and Clostridium difficile may be found on many devices, and may result in a user illness or bacterial transmission.

### SUMMARY

The following presents a simplified summary in order to provide a basic understanding of some aspects of the disclosure. The summary may be not an extensive overview of the disclosure. It may be neither intended to identify key or critical elements of the disclosure nor to delineate the scope of the disclosure. The following summary merely presents some concepts of the disclosure in a simplified form as a prelude to the description below.

Methods, devices, and techniques are described herein for providing light for disinfecting appliances. In some examples, one or more light emitters emit light in a wavelength range of about 380 nm - 420 nm for disinfection.

An example appliance may comprise a first section, a second section, a first light emitter configured to emit a first light into the first section, and a control system configured to control, based on a characteristic of the first section, a first characteristic of the first light in the first section. The control system may be further configured to control a second characteristic of a second light in the second section. The first characteristic of the first light in the first section may be independent of the second characteristic of the second light in the second section. The first light may have a first peak wavelength in a first wavelength range of 380 nm - 420 nm.

An example appliance may comprise a first section, a second section, a first light emitter configured to emit first light having a first peak wavelength in a first wavelength range of 380 nm - 420 nm into the first section, a second light emitter configured to emit second light into the second section, and a sensor. An example method of controlling light in an interior of the appliance may comprise receiving, from the sensor, sensor data, where the sensor data may comprise an indication of a characteristic of contents of the first section. The example method may further comprise determining, based on the characteristic of contents of the first section, a dosage of the first light to be provided to the first section over a period of time. The example method may further comprise emitting, using the first light emitter, a first radiant flux of the first light over the period of time to provide the determined dosage, where the first radiant flux of the first light is independent of a second radiant flux of the second light.

An example device may comprise one or more light emitters, a sensor configured to determine an occupancy in the first section of the appliance and a control system. The one or more light emitters may be configured to emit a first light, having a first peak wavelength in a first wavelength range of 380 nm - 420 nm, into a section of an appliance. The one or more light emitters may be configured to emit a second light, having a peak wavelength in a second wavelength range of 420 nm - 495 nm, into the section of the appliance. The control system may be configured to control a first radiant flux of the first light and a second radiant flux of the second light in at least the section of the appliance based on the determined occupancy.

### BRIEF DESCRIPTION OF THE DRAWINGS

Examples herein will be described in detail, with reference to the following figures, wherein like designations denote like elements.

FIG. 1 shows an example appliance with disinfection capabilities.

FIG. 2 shows another example appliance with disinfection capabilities, in accordance with one or more examples disclosed herein.

FIG. 3 shows another example appliance with disinfection capabilities, in accordance with one or more examples disclosed herein.

FIGS. 4A-4C show another example appliance with disinfection capabilities, in accordance with one or more examples disclosed herein.

FIGS. 5A-5C show another example appliance with disinfection capabilities, in accordance with one or more examples disclosed herein.

FIG. 6 shows an example method for controlling light in an appliance, in accordance with one or more examples disclosed herein.

FIG. 7 shows an example method for controlling light in an example, in accordance with one or more examples described herein.

FIG. 8 shows an example computing device, that may be used for generation and/or control of light for disinfection in appliances, in accordance with one or more examples disclosed herein.

### DETAILED DESCRIPTION

In the following description of the various embodiments, reference is made to the accompanying drawings, which form a part hereof, and in which is shown by way of illustration, various embodiments of the disclosure that may be practiced. It is to be understood that other embodiments may be utilized.

Various household appliances are susceptible to bacterial contamination. Appliances such as refrigerators, for example, may contain a level of bacteria that is above a level that may be considered to be safe. Low temperatures of refrigerators may only inhibit bacteria growth in tandem with regular cleaning. In absence of regular cleaning, bacteria may establish and propagate within the refrigerator. This may result in food spoilage, bad odors, illness, etc.

Disinfection (e.g., disinfection of surfaces in various environments) may be accomplished using different techniques. One technique may be manual cleaning with disinfecting chemical cleaners or soaps. Chemical cleaners may provide only intermittent disinfection, thereby allowing harmful microorganisms to build up between cleanings. Another technique may be ultraviolet (UV) light exposure. For example, some disinfecting systems may transmit UV light onto surfaces for disinfection. Exposure to UV light may be harmful for humans and animals, and appropriate steps may be needed to minimize such exposure. UV light may be turned off, for example, when exposure (e.g., to a user) is anticipated. UV light disinfection systems may involve complex controls to prevent direct exposure to humans to facilitate such safety mechanisms. Exposure to UV light may degrade plastics (e.g., as used in refrigerator interiors, disposable food packaging, plastic storage containers, etc.). UV may also have adverse effects on food. The adverse effects may include discoloration of fresh foods (e.g., meat), inactivation of various desirable biological molecules in fresh food (e.g., when UV light is used at high intensities), etc.

Wavelengths of visible light in the violet range of spectrum (e.g., 380 nanometers (nm) -420 nm), or a specific wavelength of violet light (e.g., 405 nm), may have a lethal effect on microorganisms such as bacteria, yeast, mold, and fungi. E. coli, Salmonella, MRSA, and Clostridium Difficile, for example, may be susceptible to 380 nm - 420 nm light. Such wavelengths may initiate a photoreaction with porphyrin molecules found in microorganisms. The porphyrin molecules may be photoactivated and may react with other cellular components to produce Reactive Oxygen Species (ROS). The ROS may cause irreparable cell damage and eventually destroy, kill, or otherwise inactivate the microorganism. This technique may be completely safe for human exposure because humans, plants, and/or animals do not contain the same porphyrin molecules.

In some examples, inactivation, in relation to microorganism death, may correspond to control and/or reduction in microorganism colonies or individual cells on exposure to disinfecting light for a certain duration. Light may be utilized for inactivation of bacterial pathogens with a peak wavelength of light, or in some examples, multiple peak wavelengths, in a wavelength range of approximately 380 nm to 420 nm. For example, approximately 405 nm light may be used as the peak wavelength. Any wavelength within a wavelength range of 380 nm to 420 nm may be utilized, and a peak wavelength may comprise a specific wavelength within the wavelength range (e.g., with reasonable variations, such as ± 5 nm, ± 10 nm, etc.). Other values and ranges disclosed herein may be used with variations of approximately 10% (or any other percentage) of the disclosed values.

Example methods, devices, and systems described herein use visible light (e.g., 380 nm -420 nm wavelength light, and/or a specific wavelength in the wavelength range) for disinfection. Visible light disinfection may be used for continuous, efficient, and effective decontamination of various surfaces, device, and/or appliances. Visible light disinfection may be simultaneous with normal operation and without interruption of other functions of the devices and/or appliances. Daily and/or terminal cleaning procedures may be supplemented with visible light disinfection to maintain cleanliness between such cleaning procedures. Visible light disinfection may be used, for example, to combat any new sources of contamination and/or to reduce growth rates of microorganisms that may be left behind after typical cleaning procedures.

Exposure to light (e.g., in a 380 nm - 420 nm wavelength range, or a specific wavelength in the wavelength range) with an irradiance that is greater than or equal to a minimum irradiance may cause microbial inactivation (e.g., disinfection). Light at an irradiance that is greater than or equal to a minimum irradiance of, for example, 0.02 mW/cm² may cause microbial inactivation on a surface over time. Light at an irradiance of, for example, 0.05 mW/cm² may inactivate microorganisms on a surface, but higher values (e.g., 0.1 mW/cm², 0.5 mW/cm², 1 mW/cm², or 2 mW/cm²) may be used for faster inactivation. Even higher irradiances (e.g., 3 to 10 mW/cm²) may be used for microbial inactivation over shorter periods of time. Light for microbial inactivation may include radiometric energy that is sufficient to inactivate at least one bacterial population, or a plurality of bacterial populations.

Dosage (measured in Joules/cm², or J/cm²) may be a metric for determining an appropriate irradiance for microbial inactivation over a period of time. Table 1 below shows example correlations between irradiance, in mW/cm², and dosage, in J/cm², based on different exposure times. These values are examples and many others may be possible.

TABLE 1

Example correlations between irradiance and dosage

| Irradiance (mW/cm²) | Exposure Time (hours) | Dosage (Joules/cm²) |
|---|---|---|
| 0.02 | 1 | 0.072 |
| 0.02 | 24 | 1.728 |
| 0.02 | 250 | 18 |
| 0.02 | 500 | 36 |
| 0.02 | 1000 | 72 |
| 0.05 | 1 | 0.18 |
| 0.05 | 24 | 4.32 |
| 0.05 | 250 | 45 |
| 0.05 | 500 | 90 |
| 0.05 | 1000 | 180 |
| 0.1 | 1 | 0.36 |
| 0.1 | 24 | 8.64 |
| 0.1 | 250 | 90 |
| 0.1 | 500 | 180 |
| 0.1 | 1000 | 360 |
| 0.5 | 1 | 1.8 |
| 0.5 | 24 | 43.2 |
| 0.5 | 250 | 450 |
| 0.5 | 500 | 900 |
| 0.5 | 1000 | 1800 |
| 1 | 1 | 3.6 |
| 1 | 24 | 86.4 |
| 1 | 250 | 900 |
| 1 | 500 | 1800 |
| 1 | 1000 | 3600 |

Microbial inactivation may comprise a target reduction in bacterial population(s) (e.g., 1-Log₁₀ reduction, 2-Log₁₀ reduction, 99% reduction, or the like). Table 2 shows the different dosages recommended for the inactivation (measured as 1- Log₁₀ reduction in population) of different bacterial species using narrow spectrum 405 nm light. Example dosages and other calculations shown herein may be determined based on laboratory settings. Real world applications may require dosages that may differ from example laboratory data. Other dosages of 380 nm -420 nm (e.g., 405 nm) light may be used with other bacteria not listed below.

TABLE 2

Recommended dosages for inactivation of different bacterial species

| Organism | Recommended Dose (J/cm²) for 1-Log₁₀ Reduction in Bacteria |
|---|---|
| Staphylococcus aureus | 20 |
| MRSA | 20 |
| Pseudomonas aeruginosa | 45 |
| Escherichia coli | 80 |
| Enterococcus faecalis | 90 |

Equation 1 may be used in order to determine irradiance, dosage, or time using one or more data points from Table 1 and/or Table 2:

$$\frac{I r r a d i a n c e \left(\frac{m W}{c {m}^{2}}\right)}{1000} ∗ T i m e \left(s\right) = D o s a g e \left(\frac{J}{c {m}^{2}}\right)$$

Irradiance may be determined based on dosage and time (e.g., using Equation 1). An irradiance of approximately 1 mW/cm² may be needed for inactivation, for example, if a dosage of 30 J/cm² is required over 8 hours. A smaller irradiance of approximately 0.3 mW/cm² may be sufficient for inactivation, for example, if a dosage of 50 J/cm² is required over 48 hours. Irradiance may be adjusted by adjusting radiant flux of a light emitter.

Exposure time may be determined based on irradiance and dosage (e.g., using Equation 1). A target bacterial population may require a particular dosage (e.g., 20 J/cm²) for inactivation, and a light emitting device may be configured to generate disinfecting light corresponding to a specific irradiance (e.g., 0.05 mW/cm²). In this scenario, a minimum exposure time of approximately 4.6 days may be required to achieve the particular dosage (e.g., 20 J/cm²).

Dosage values may be based on a target reduction in bacterial population (e.g., 1-Log₁₀ reduction, 2-Log₁₀ reduction, 99% reduction, or the like). A larger degree of reduction may require a larger dosage. Disinfecting light may be continuously or intermittently applied to keep the bacterial population under control, for example, after a target reduction in bacterial population is attained.

Different colors, wavelengths, and/or wavelength ranges of light may be utilized for inactivation, provided that a portion (e.g., 20%, 40%, or the like) of the total spectral power and/or total spectral energy is within a wavelength range of 380 nm - 420 nm. A white light containing energy across the visible light spectrum within the wavelength range of 380 nm - 750 nm, with, for example, at least 20% of total energy within a wavelength range 380 nm - 420 nm, may be used for disinfection purposes. Light emitted from a light emitter may be white, may have a color rendering index (CRI) value of at least 70, may have a correlated color temperature (CCT) between approximately 2,500 K and 5,000 K, and/or may have 10% to 44% of spectral energy and/or spectral power in a 380 nm - 420 nm wavelength range. Other colors of light (e.g., blue, green, red, and/or the like), with a portion of spectral energy and/or spectral power within a wavelength range of 380 nm -420 nm that is greater than a threshold (e.g., 20%), may also be used for disinfection.

Larger spaces (e.g., entire rooms) may be disinfected as part of general illumination systems. A UV light, violet light, or white light comprising a certain proportion of disinfecting light, for example, may be used. General overhead illumination may not be applicable for appliances because light may not necessarily be able to make sufficient contact with infected surfaces within an appliance (e.g., inside of a washing machine, refrigerator, and/or the like). Other challenges for providing disinfection to appliances may include designing a disinfection system for interior and/or exterior surfaces with irregular shapes, and/or comprising objects that are not originally intended to have such a disinfection system associated therewith.

As described herein, safe visible light disinfection may be provided for appliances to control the growth of harmful microorganisms. Visible light disinfection may prevent illness in humans as well as control other negative effects of microorganisms such as odor or visually unappealing mold and/or fungi. Appliances such as, for example, refrigerators, freezers, ice makers, ice dispensers, food warmers, display cases, salad bars, waste disposal equipment, food storage areas, dishwashers, sinks, washing machines, dryers, garbage disposals, trash compactors, etc., may benefit from visible light disinfection as described herein. In various examples, disinfecting light may be used alone, or in combination with white light.

Certain foods (e.g., cheese, fresh foods such as fruits and vegetables) may contain photosensitizers that absorb disinfecting light within the visible light range (e.g., 380 nm -420 nm wavelength range). Exposure to such wavelengths may result in photo-degradation. Various examples described herein use techniques for reducing photo-degradation of foods that may be caused by disinfecting light.

As described herein, light used for disinfection may be continuous or intermittent. An object or a surface intended to be disinfected may be continuously illuminated. An object or surface may be illuminated with disinfecting light for a first fraction of time (e.g., 80% of the time) and not illuminated with disinfecting light for a second fraction of time (e.g., 20% of the time). An object or a surface may be illuminated by disinfecting light, for example, if the object or the surface is not being interacted with (e.g., not being used) by a user. An object or a surface may not be illuminated by disinfecting light, for example, if the object or the surface is being interacted with (e.g., being used) by a user. For example, disinfecting light may be deactivated if a user opens an appliance door (e.g., a refrigerator door), a user uses a toilet, a user opens a garbage can, etc.

As described herein, a control system (e.g., integrated with an appliance) may determine that a minimum disinfecting light dosage (e.g., over a certain period of time) has been met for disinfecting purposes and deactivate the disinfecting light. Deactivating the disinfecting light may save energy, and/or prevent photo-degradation (e.g., photo-degradation of vegetables in a refrigeration). The disinfecting light may remain deactivated, for example, for a period of time (e.g., a predefined period, a user-configured period of time). For example, disinfecting light may be turned off 30% of time in a specific time period (e.g., 24 hours). In this example, the disinfecting light may remain activated for 16.8 hours out of 24 hours. Other similar ratios may be possible. Various controls systems described herein apply/adjust exposure time, radiant flux of a light emitter, irradiance, color, wavelength, dosage, etc. of applied light based on various considerations described herein.

Various methods, devices, and systems described herein may use, for example, light in different wavelength ranges (e.g., 380 nm - 420 nm, 420 nm - 500 nm) to inactivate insects. In various examples described herein, light at a specified wavelength or wavelength range may correspond to light which has a maximum emitted energy/power/energy spectral density/power spectral density approximately at the specified wavelength or within the specified wavelength range, with reasonable variations (e.g., ± 5 nm, ± 10 nm, etc.).

Many different appliances may be integrated with visible disinfecting light (e.g., light in a 380 nm - 420 nm wavelength range) in order to inactivate microorganisms and/or to prevent effects such as illness or unpleasant odor. The teachings of this disclosure are not limited to specific appliances and devices described herein and may be applicable to similar appliances and devices.

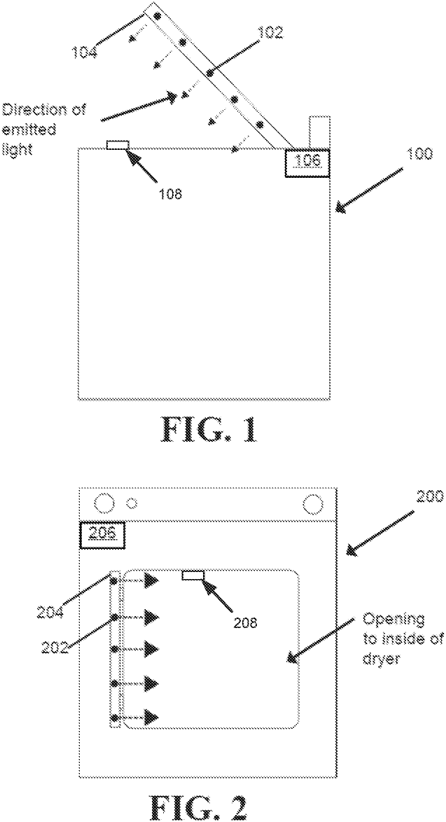

FIG. 1 shows an example appliance 100 with disinfection capabilities. The appliance 100 may be, for example, a washing machine. The appliance 100 may comprise disinfecting light emitters 102 integrated into a bottom of a lid/door 104. Additionally, or alternatively, disinfecting light emitters may be placed in the interior of the appliance 100. The appliance 100 may comprise disinfecting light emitters, for example, on a drum (e.g., on an inside surface of the drum) of the washing machine. Light from the light emitters 102 may be directed to interior surfaces of the appliance 100. The light emitters 102 may be positioned in a manner such that light emitted by the light emitters 102 is directed to interior surfaces of the appliance 100 when the door/lid 104 is closed. The light emitters 102 may emit light in a 380 nm - 420 nm wavelength range (in a portion of the wavelength range, a specific wavelength in the wavelength range, or having a peak wavelength in the wavelength range).

The appliance 100 may comprise a control system 106. The control system 106 may control disinfecting light, for example, based on a change of status of the appliance 100 (e.g., based on whether the lid/door 104 is open or closed. The control system 106 may cause and/or adjust illumination, by the disinfecting light emitters 102, for example, based on whether the lid/door 104 is open or closed. The control system 106 may activate disinfecting light, for example, if the lid/door 104 is closed, and deactivate disinfecting light, for example, if the lid/door 104 is open. The control system 106 may determine, using a sensor 108 (e.g., a motion sensor, a switch, a light sensor, etc.), whether the door 104 is open or closed. Disinfection may be utilized in between and/or during wash cycles. Disinfecting light may be activated, for example, during a wash cycle (e.g., during rinsing, during spinning, etc.).

FIG. 2 shows an example appliance 200 with disinfection capabilities. The appliance 200 may comprise features described above with respect to the appliance 100. The appliance 200 may be, for example, a clothes dryer. The appliance 200 may comprise disinfecting light emitters 202 integrated with a door 204 (e.g., on an inside surface of the door 204). Additionally, or alternatively, disinfecting light emitters may be integrated in the interior of the appliance 200. The appliance 200 may comprise disinfecting light emitters, for example, on a drum (e.g., on an inside surface of the drum) of the clothes dryer. The light emitters 202 may be positioned in a manner such that light emitted by the light emitters 202 is directed to interior surfaces of the appliance 200 when the door 204 is closed. The light emitters 202 may emit light in a 380 nm - 420 nm wavelength range (or in a portion of the wavelength range, or a specific wavelength in the wavelength range).

The appliance 200 may comprise a control system 206. The control system 206 may control disinfecting light, for example, based on a change of status of the appliance 200. The control system may cause and/or adjust illumination, by the disinfecting light emitters 202, for example, based on whether the door 204 is open or closed. The control system 206 may activate disinfecting light, for example, if the door 204 is closed, and deactivate disinfecting light, for example, if the door 204 is open. The control system 206 may determine, using a sensor 208 (e.g., a motion sensor, a switch, a light sensor, etc.), whether the door 204 is open or closed. Disinfection may be utilized in between and/or during cycles.

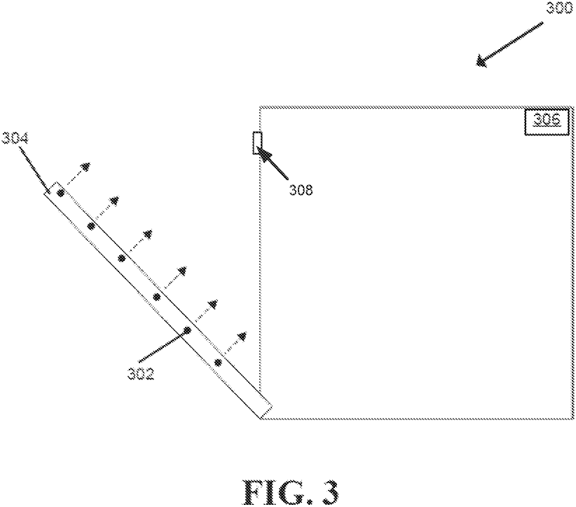

FIG. 3 shows an example appliance 300 with disinfection capabilities. The appliance 300 may comprise features described above with respect to the appliance 100 and/or the appliance 200. The appliance 300 may be, for example, a dishwasher. The appliance 300 may comprise disinfecting light emitters 302 integrated with a door 304 (e.g., on an inside surface of the door 304). Additionally, or alternatively, disinfecting light emitters may be integrated in the interior of the appliance 300. Light emitters 302 may be positioned in a manner such that light emitted by the light emitters 302 is directed to interior surfaces of the appliance 300 when the door 304 is closed. The light emitters 302 may emit light in a 380 nm - 420 nm wavelength range (or in a portion of the wavelength range, or a specific wavelength in the wavelength range).

The appliance 300 may comprise a control system 306. The control system 306 may control disinfecting light, for example, based on a change of status of the appliance 300. The control system may cause and/or adjust illumination, by the disinfecting light emitters 302, for example, based on whether the door 304 is open or closed. The control system 306 may activate disinfecting light, for example, if the door 304 is closed, and deactivate disinfecting light, for example, if the door 304 is open. The control system 306 may determine, using a sensor 308 (e.g., a motion sensor, a switch, a light sensor, etc.), whether the door 304 is open or closed. Disinfection may be utilized in between and/or during wash cycles. Disinfecting light may be activated, for example, during a wash cycle (e.g., during rinsing). Disinfecting light may be activated, for example, during a drying process.

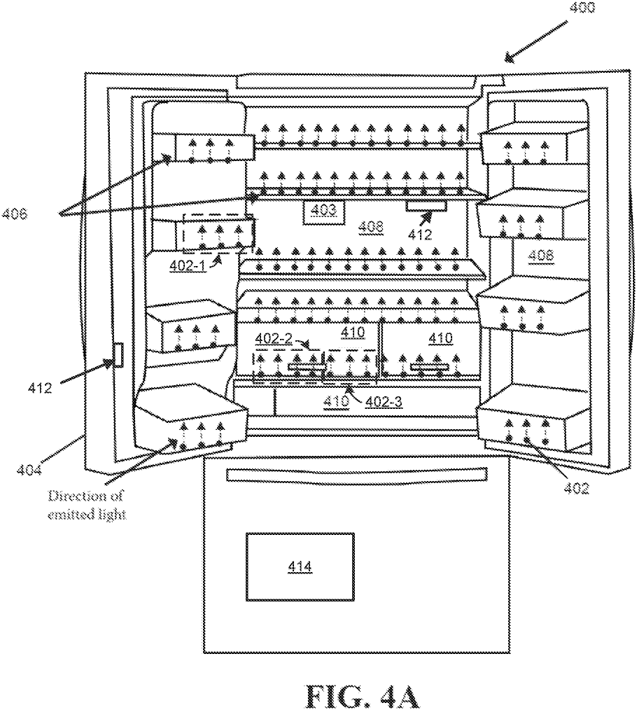

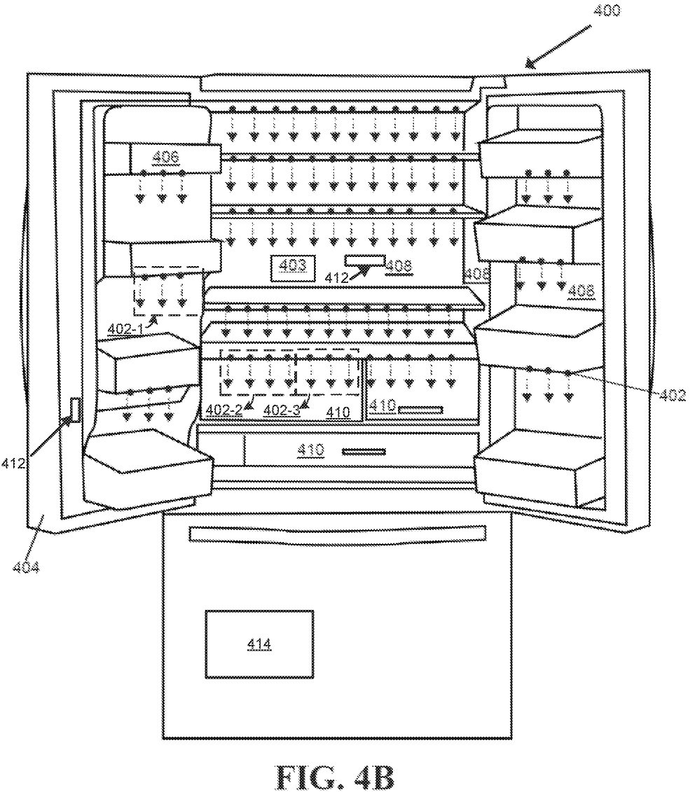

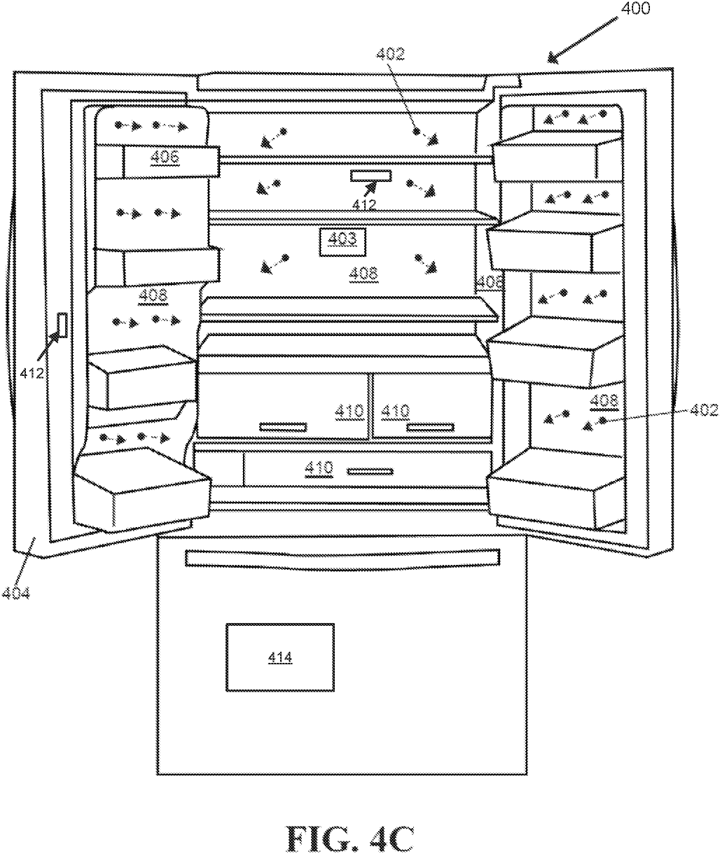

FIGS. 4A-4C show an example appliance 400 with disinfection capabilities. The appliance 400 may comprise features described above with respect to the appliance 100, the appliance 200, and/or the appliance 300. The appliance 400 may be a refrigerator. The appliance 400 may comprise light emitters 402, doors 404, shelving 406, and drawers 410. The appliance 400 may further comprise a control system 403. The control system 403 may adjust one or more parameters (e.g., dosage, time of activation, color, wavelength, intensity, radiant flux, and/or irradiance) of light, for example, based on measurements received from one or more sensors 412. The control system 403 may be configured to control parameters of light in different wavelength ranges. The control system 403 may adjust the one or more parameters based on user input. The control system 403 may be configured to control operation of the light emitters 402 to produce different intensities (e.g., less than 100 foot-candles or about 1000 lumen/m² (lux), less than 10 foot-candles or about 100 lux, etc.). The appliance 400 may comprise a user interface 414 (e.g., a touch screen monitor) that may be used to control operation of the appliance 400. In an example, a device may comprise (e.g., in an integrated package) one or more of the light emitters 402, the control system 403, and the one or more sensors 412. The device may be configured for attachment/placement within a section of the appliance 400.

The light emitters 402 may emit light (e.g., disinfecting light, white light, etc.) with at least a portion of spectral energy or spectral power in a wavelength range of 380 nm - 420 nm. The light emitters 402 may be integrated with the appliance 400 in one or more locations (e.g., in the doors 404, shelving 406, and/or drawers 410). The shelving 406 may be transparent or translucent. Transparent or translucent shelving may allow better disinfecting light transmission within the appliance 400. At least some sections/parts of an interior of the appliance may comprise opaque and/or reflective materials to enable higher light intensities (e.g., in different sections of the appliance 400).

The light emitters 402 may be integrated into the shelving 406 and/or drawers 410, and may direct light upwards (e.g., FIG. 4A). The light emitters 402 may be integrated into shelving 406 and/or drawers 410, and may direct light downwards (e.g., FIG. 4B). The light emitters 402 may be integrated into the doors 404 and may direct light perpendicular to an inside surface of the doors 404 in a manner such that light is directed onto interior surfaces of the appliance 400 when the doors 404 are closed (e.g., FIG. 4C). The light emitters 402 may be integrated on side walls 408 (e.g., a back surface) of the appliance 400 in a manner such that light is directed towards the shelving 406, drawers 410, and/or interior surfaces of the doors 404. Other lighting arrangements may be possible for the appliance 400.

The light emitters 402 may be integrated or retrofitted such that the appropriate amount of disinfecting energy is incident on surfaces required to be disinfected. Appliances may either be integrated with the light emitters 402 during new product design/manufacturing or have light emitters 402 added to them after the fact as a retrofit solution.

The shelving 406 and/or drawers 410 may be internally illuminated with the light emitters 402. The light emitters 402 may be integrated on the inside of the shelving 406 and/or drawers 410. The light emitters 402 may direct light out in all directions. The light emitters 402 may direct light through the shelving 406 (e.g., if the shelving 406 is transparent or translucent). Light from the light emitters 402 may be contained within the shelving 406 and/or drawers 410. The shelving 406 and/or drawers 410 may be transparent or translucent such that light from the light emitters 402, or a portion of the light, may pass through. The shelving 406 and/or drawers 410 may be configured to absorb, for example, only a portion (e.g., less than 10%, or any other percentage) of any disinfecting energy emitted by the one or more light emitters 402. Light may be directed and/or reflected (e.g., using a control system) inside the appliance 400 to hit more surfaces. The light emitters 402 may be configured to provide an appropriate amount of disinfecting light intensity to initiate inactivation of microorganisms (e.g., 100 lux, 200 lux, 500 lux, or any other value).

The light emitters 402 may be configured to emit various wavelengths of light. The light emitters 402 may be configured to emit white light. At least some of the light emitters 402 may be configured to emit light in a visible light wavelength range (or a subset thereof) of 380 nm-750 nm. At least some of the light emitters 402 may be configured to emit light in a wavelength range of 420 nm - 495 nm. At least some of the light emitters 402 may be configured to emit light with at least a portion thereof in the wavelength range of 380 -420 nm (e.g., 405 nm), and with an irradiance and/or dosage sufficient to initiate inactivation of microorganisms. At least some of the light emitters 402 may be configured to provide disinfecting light in accordance with Tables 1 and 2, and Equation 1 above.

Different sections of the refrigerator 400 may be configured with different wavelengths of light. One or more sections of the refrigerator 400 may be configured with a light that does not cause photo-degradation. One or more sections of the refrigerator 400 may be configured with a light (e.g., 420 nm - 495 nm wavelength range) that encourages photosynthesis and increases lifetime of stored produce. A first section of the refrigerator 400 (e.g., the shelving 406) may be configured with light in a first wavelength range (e.g., 380 nm - 420 nm) and a second section of the refrigerator 400 (e.g., the drawers 410) may be configured with light in a second wavelength range (e.g., a wavelength range that does not cause photo-degradation). The different sections may be used for storing different items. The second section may be used, for example, for storing fruits, vegetables, and/or meat.

The different wavelength ranges may be configured using different types of light emitters. The different wavelength ranges may be configured using a tunable light emitter (e.g., tunable light emitting diode). A first section of the refrigerator 400 (e.g., the shelving 406) may be configured with light emitters (e.g., light emitters 402-1) emitting light in a first wavelength range (e.g., 380 nm - 420 nm) and a second section of the refrigerator 400 (e.g., the drawers 410) may be configured with light emitters (e.g., light emitters 402-2) emitting light in a second wavelength range. The second wavelength range may be different from the first wavelength range. In an example, the second wavelength range may be 420 nm - 495 nm. In various examples, light emitters in the first section may be configured to emit light into the first section and light emitters in the second section may be configured to emit light into the second section. In various examples, light emitters in the first section may be further configured to emit light into the second section.

The different wavelength ranges may be configured using light conversion and/or filtering techniques. Various sections of the appliance 400 may comprise light converting materials and/or light filtering materials. Mechanical elements of a section (e.g., side walls of the section, floors of the section, etc.) may be configured to be transparent or translucent, and may be coated (and/or embedded) with light converting materials and/or light filtering materials. Light filtering materials may be materials that are configured to filter out (e.g., block) one or more ranges of wavelengths. A first section of the refrigerator 400 (e.g., the shelving 406) may comprise a light filtering material that is configured to allow light in a first wavelength range (e.g., 380 nm - 420 nm) to pass through, and/or a second section of the refrigerator 400 (e.g., the drawers 410) may comprise a light filtering material that is configured to allow light in a second wavelength range (e.g., a wavelength range that does not cause photo-degradation, 420 nm - 495 nm). Light filtering materials may comprise, for example, tinted plastic and/or other translucent materials.

Light emitted by the light emitters 402 may be converted and/or filtered by such materials and may be incident on an interior of the section. In an example, different sections of the refrigerator 400 may be configured with different light converting materials that produce corresponding different wavelength ranges. In an example, the light emitters 402 may be configured to emit a single wavelength of light. A first section (e.g., the shelving 406) may be coated with light converting materials configured to convert the light to light in a first wavelength range (e.g., 380 nm - 420 nm). A second section (e.g., the drawers 410) may be coated with light converting materials configured to convert the light to light in a second wavelength range (e.g., 420 nm - 495 nm).

The control system 403 may control one or more parameters (e.g., dosage, time of activation, color, wavelength, intensity, radiant flux, and/or irradiance) of light emitted by the light emitters 402 using on one or more techniques described herein. The control system 403 may control operation of light emitters 403 and adjust various parameters (e.g., dosage, time of activation, color, wavelength, intensity, radiant flux, and/or irradiance) of light in different wavelength ranges using on one or more techniques described herein. The control system 403 may adjust the one or more parameters based on measurements obtained from one or more sensors (e.g., the sensors 412). The sensors 412 may comprise, for example, motion sensors, voice sensors, light beam sensors, infrared sensors, odor sensors, magnetic proximity sensors, capacitive touch sensors, light sensors, infrared sensors, cameras, ultrasonic sensors, weight sensors, limit switches, irradiance sensors, intensity sensors, etc.

The control system 403 may be configured to control parameters of light (e.g., dosage, time of activation, color, wavelength, intensity, radiant flux, and/or irradiance) in at least some sections of the appliance 400. The control system 403 may be configured to independently control the parameters of light emitted in different sections (e.g., the drawers 410, the shelving 406, etc.). For example, parameters of light in a section may be based on characteristics of the section and/or contents in the section. Parameters of light in a section may be independent of characteristics of contents in other sections and/or parameters of light used for other sections. The control system 403 may use one or more techniques described herein to independently control the parameters of light in different sections.

The control system 403 may adjust one or more parameters of light within the appliance 400. The control system 403 may adjust the one or more parameters, for example, based on a status (or change thereof) of the appliance 400 and/or contents of the appliance 400. A change of status may correspond to user interaction with the appliance 400 (e.g., a user opening or closing a door/lid, user providing a voice command to the appliance 400), a desired light dosage being attained, properties associated with items stored in the appliance 400, etc. The sensors 412 may be used to measure a change in status. The control system 403 may adjust one or more parameters of light emitted by light emitters 402 based on the change in status.

The sensors 412 (e.g., a motion sensor, touch sensor, and/or the like) may be used to detect user interaction with the appliance 400 (e.g., an appliance door being open or closed, an appliance handle being touched, etc.). For example, the control system 403 may determine whether the doors are open or closed using measurements from the sensors 412. The control system 403 may activate disinfecting light based on a determination that the door 404 is closed, deactivate the disinfecting light based on a determination that the door 404 is open, and/or replace the disinfecting light with illuminating white light based on a determination that the door 404 is open. The control system may 403, for example, activate the illuminating white light and deactivate the disinfecting light when the door 404 is opened. The control system may replace disinfecting light (e.g., 405 nm light) in the appliance 400 with disinfecting white light based on detecting a user interaction. Disinfecting white light may comprise, for example, light in a visible light wavelength range of 380 nm - 750 nm, with at least a portion of its spectral energy (e.g., greater than 20%) in a wavelength range of 380 nm - 420 nm.

The sensors 412 may determine aspects of the appliance 400 and/or characteristics of contents in the appliance 400. The sensors 412 may determine, for example, weight of a section (e.g., container) within an appliance 400, occupancy of a section within the appliance 400, a quantity of items in a section within the appliance 400, a number of times the door 404 has been opened, a duration for which the door 404 is open, whether a handle of the appliance 400 is being touched, whether a food item has been in the appliance 400 for more than a threshold amount of time, whether the light emitters 402 have been on for more than a threshold amount of time, etc. The control system 403 may appropriately control light emitted by the light emitters 402 based on measurements from the sensors 412.

The control system 403 may adjust dosage of light emitted into the drawer 410, for example, in proportion to the weight of the drawer 410 or a quantity of items in the drawer 410. Weight sensors and/or image sensors may be used to determine a quantity of items in the drawer 410. The control system 403 may adjust dosage of disinfecting light emitted into the appliance 400, for example, in proportion to a number of times the door 404 has been opened. The control system 403 may adjust dosage of disinfecting light emitted into the appliance 400, for example, in proportion to a duration for which the door 404 is open. The control system 403 may increase dosage of disinfecting light emitted into the drawer 410, for example, if items in the drawer 410 have been kept in the drawer 410 for more than a threshold amount of time. Sensors such as motion sensors, limit switches, and/or the like may be used to determine status of the door 404. Timers may be used to determine duration (e.g., duration for which the door is open).

The control system 403 may determine, using the sensors 412, parameters of disinfecting light (e.g., irradiance, radiant flux, exposure time, dosage, energy, etc.) at one or more locations (e.g., midpoints of shelving 406, drawers 410, and/or on various surfaces). The control system 403 may adjust intensities of light from the light emitters 402 and/or activation times of the light emitters 402 based on measurements from the sensors 412. The control system 403 may, for example, compare the measured parameters of disinfecting light to corresponding threshold(s). The control system 403 may adjust light from the light emitters 402, based on the comparisons, to ensure that disinfecting light parameters are at a level sufficient to inactivate microorganisms at different locations of the appliance 400.

The control system 403 may control parameters of disinfecting light (e.g., dosage, exposure time, radiant flux, wavelength, energy, and/or irradiance) to reduce possible negative effects of light exposure to fresh food (e.g., photo-degradation). The control system 403, for example, may comprise or may be in communication with sensors 412 (e.g., irradiance sensors, intensity sensors, etc.) and determine various parameters of disinfecting light (e.g., irradiance, exposure time, dosage, energy, etc.) at one or more locations within the appliance 400. The control system 403 may compare the determined parameters of disinfecting light to photo-degradation threshold(s) above which photo-degradation may occur. The control system 403 may, based on the comparisons, adjust disinfecting light from the light emitters 402. The control system 403 may, for example, adjust the disinfecting light (e.g., deactivate light emitters 402, reduce disinfecting light intensity, etc.) to ensure the disinfecting light does not cause photo-degradation. The control system 403 may, for example, adjust the disinfecting light such that an irradiance of the disinfecting light is below the photo-degradation threshold. The control system 403 may be configured to control the parameters of disinfecting light in a manner such that the disinfecting light does not degrade foods, but provide disinfecting light dosages that are sufficient to disinfect surfaces within the appliance 400.

At least some sections of an appliance (e.g., the appliance 400) may be configured to remain “dark” (e.g., not illuminated by disinfecting light or intermittently illuminated by disinfecting light) and may be used for items that are more likely to be degraded by disinfecting light. In some examples, the control system may be configured to illuminate some sections (e.g., refrigerator drawers such as the crisper drawer, the drawers 410, etc.) with disinfecting light in the range of 380 - 420 nm while other sections of the refrigerator remain “dark.”

The control system 403 may be configured to apply blue light in the range of 420 - 495 nm to encourage photosynthesis and increase the lifetime of produce. The blue light may be used, for example, in “dark” sections of the refrigerator 400.

In an example, the sensors 412 may comprise image sensors (e.g., cameras) and the control system may determine, based on measurements from the image sensors, an identity/type of contents in a section of the refrigerator 400. The control system 403 may analyze an image from an image sensor to determine a color of the contents, shape of the contents, outline of the contents, and/or other parameters. The controller may use pre-trained classifiers and/or machine learning tools to identify contents in the section. Determining the color of the contents may comprise determining a color that is associated with a maximum number of pixels in an image captured by the image sensor. The controller may determine that the contents are leafy green vegetables, for example, if the color of the contents is determined to be green (e.g., wavelength range of 495 nm - 570 nm). The controller may determine that the contents are not leafy green vegetables, for example, if the color is determined to be any color other than green.

The control system 403 may apply blue light (e.g., 420 nm - 500 nm wavelength range using the light emitters 402-2) to the section based on the determination that the contents are leafy green vegetables. The control system may, for example, apply a predetermined dosage of the blue light. The control system may deactivate the disinfecting light, for example, based on a determination that the contents are leafy green vegetables. The control system 403 may apply disinfecting light (e.g., 380 nm - 420 nm wavelength range using the light emitters 402-1) to the section based on a determination that the contents are not leafy green vegetables. The control system may deactivate the blue light, for example, based on a determination that the contents are not leafy green vegetables. In at least some examples, the control system may apply the blue light and the disinfecting light at the same time.

The control system 403 may use a first disinfecting light dosage in a first section and a second disinfecting light dosage in a second section. The first disinfecting light dosage may be lower than the second disinfecting light dosage. The control system may use a lower photo-degradation threshold in the first section than in the second section. The drawers 410 may be used, for example, for storage of fresh foods and may be configured with a disinfecting light dosage that is lower than a disinfecting light dosage used for the shelving 408.

The control system 403 may vary parameters of disinfecting light (e.g., dosage, radiant flux, time, wavelength, and/or irradiance) based on power consumption considerations. The control system 403 may adjust the light to respond to energy consumption thresholds/limits. The control system 403 may, for example, deactivate disinfecting light when an energy consumption limit is reached. The control system 403 may use algorithms to predict if a threshold/limit is going to be exceeded. The control system 403 may collect historical data related to usage of the appliance 400, make a prediction of energy consumption based on the historical data, and adjust the lighting to ensure a threshold/limit is not exceeded. The control system 403 may, for example, lower disinfecting light dosage in response to anticipating that an energy consumption limit is being approached. The threshold/limit may be defined, for example, for a predetermined time period (e.g., 24 hours), and the control system 403 may continuously adjust the lighting to ensure the threshold/limit is not exceeded over the predetermined time period.

The control system 403 may be configured to control a dosage of light by adjusting a duration of exposure based on one or more considerations described herein. The control system 403 may alternate between activating and deactivating the light emitters 402. The control system 403 may be configured to, for example, alternately activate the light emitters 402 for a first duration of time (e.g., 24 hours) and deactivate the light emitters 402 for a second duration of time (e.g., 1 hour).

The control system 403 may be configured with a disinfecting cycle. The disinfecting cycle may be initiated by a user through the user interface 414. Light emitters may be adjusted to operate at a higher level of energy and/or irradiance for a specified period of time, for example, when the disinfecting cycle is activated. A user may activate a disinfecting cycle, for example, during off peak energy times when energy (e.g., electricity) costs are low. The control system 403 may automatically activate a disinfecting cycle, for example, in response to determining that energy costs are low. The control system 403 may communicate, for example, with a server (e.g., via internet) to determine current energy costs and activate the disinfecting cycle based on the energy costs. Activating the disinfecting cycle only for a specified period of time may reduce energy usage outside of the disinfecting cycle while still allowing a high dose of disinfecting energy.

Intensities and/or irradiances of disinfecting light need not be the same on all the interior surfaces of an appliance. With reference to the appliance 400, for example, based on the arrangement of light emitters 402 and arrangement of objects within the appliance 400, the side walls 408 may be exposed to an irradiance that is different from an irradiance on a surface of the doors 408. The control system 403 may adjust intensities of the light emitters 402 to produce required irradiances for microbial inactivation (e.g., 0.05 mW/cm², 0.5 mW/cm², 1 mW/cm²m, 2 mW/cm², or the like) on a majority of the interior surfaces (e.g., at least 80% of the interior surfaces) in the appliance 400. Irradiances of disinfecting light on different surfaces may be measured using sensors (e.g., irradiance sensors, intensity sensors, etc.) located on the different surfaces.

The control system 403 may activate the light emitters 402 and/or increase a radiant flux of light emitters 402 in a section of the appliance 400, for example, based on presence of items in the section and/or increase in a quantity of items in the section. The control system 403 may deactivate the light emitters 402 and/or reduce a radiant flux of light emitters 402 in a section of the appliance 400, for example, based on absence of items in the section and/or reduction in a quantity of items in the section. The control system 403 may, for example, determine that an item is located in a first section (e.g., the drawers 410) of the appliance 400, and no items are located in a second section (e.g., the shelving 406) of the appliance 400. The control system 403 may apply disinfecting lighting in the first section and not apply disinfecting lighting in the second section.

The control system 403 may adjust one or more parameters (e.g., dosage, time of activation, color, wavelength, intensity, radiant flux, and/or irradiance) of light in a section based on occupancy of the section and/or a level of occupancy in the section. Occupancy in the section and/or a level of occupancy in the section may be determined using sensors such as weight sensors, motion sensors, cameras, etc. For example, higher weight may be associated with a higher level of occupancy in the section. The control system 403 may activate, based on determining that a section is occupied (e.g., the section comprises at least one item), light emitters configured to emit light in the section. The control system may be configured to apply, using the light emitters and based on the determined occupancy, a predetermined dosage of light. The control system 403 may adjust a radiant flux of the light emitters based on a level of occupancy in the section. The control system 403 may adjust a radiant flux in proportion to the level of occupancy in the section.

The control system 403 may adjust one or more parameters (e.g., dosage, time of activation, color, wavelength, intensity, radiant flux, and/or irradiance) of light emitted into the drawer 410, for example, based on motion of contents in the drawer 410. Motion of the contents and/or frequency of motion may be used as a measure of occupancy in the drawer 410. For example, the drawer 410 may be considered to be occupied if a sensor detects motion of the drawer 410 and/or motion of contents in the drawer 410. For example, greater frequency of motion may be associated with a larger level of occupancy in the drawer 410. Motion sensors, weight sensors and/or image sensors may be used to determine motion of contents in the drawer 410. The control system 403 may apply a dosage of light (e.g., into the drawer 410) based on detected motion in the drawer 410. The dosage of light may be predetermined. The dosage of light may be proportional to a frequency of motion.

The control system 403 may be configured to apply a lower radiant flux (or deactivate a light emitter) based on determining that the section is unoccupied, and a higher radiant flux (or activate a light emitter) based on determining that the section is occupied (e.g., in order to disinfect contents in the section). Deactivating the light emitter if the section is unoccupied may reduce energy consumption of the light emitter.

The control system 403 may be configured to apply a higher radiant flux based on determining that the section is unoccupied, and a lower radiant flux based on determining that the section is occupied (e.g., in order to apply disinfecting light to the surfaces, instead of/or in addition to contents of the section). Increasing the radiant flux if the section is unoccupied may enable disinfection of surfaces in the section. Reducing the radiant flux if the section is occupied may reduce photo-degradation of the contents.

The appliance 400 may comprise and/or be integrated with food recognition technology (FRT). The control system 403, for example, may use one or more sensors (e.g., infrared sensors, cameras, ultrasonic sensors, weight sensors, and/or the like) and/or user input, to determine presence of item(s) in the appliance, type(s) of item(s) in the appliance 400, locations of item(s) in the appliance 400, and/or quantity of item(s) in the appliance 400. The control system 403 may control parameters of light (e.g., intensity, dosage, radiant flux, exposure time, color, wavelength, and/or irradiance) emitted by the light emitters 402 based on measurements from the one or more sensors. The control system 403 may use FRT to independently control parameters of different types of light (e.g., disinfecting light, white light, light in different wavelength ranges, etc.) in one or more sections based on measurements from the one or more sensors. The parameters may be controlled by using different types of light emitters, different wavelengths of light, adjusting power output of light emitters, etc.

The control system 403 may activate the light emitters 402 and/or adjust parameters (e.g., wavelength, intensity, etc.) of light emitted by the light emitters 402 in specific sections of the appliance 400, for example, based on presence of items and/or type of items. The control system 403 may (e.g., using FRT, one or more sensors, or a user input, etc.) determine (e.g., identify) an item and determine a location of the item. The control system 403 may, for example, determine an item (e.g., determine a presence of the item and/or determine a type of item) in a section (e.g., the drawers 410) of the appliance 400 and may adjust an intensity of the light emitters 402 in the section, wavelength of the light emitters 402 in the section, activate the light emitters 402 in the section, and/or deactivate the light emitters 402 in the section.

The control system 403 may apply specific wavelengths of light in a section based on presence of items and/or type of items in the section. The control system 403 may apply different wavelengths of light in different sections. A section of the refrigerator may comprise different sets of light emitters that emit light in different wavelength ranges. For example, the drawers 410 may comprise the light emitters 402-2 emitting light in a first wavelength range and light emitters 402-3 emitting light in a second wavelength range. The light in the first wavelength range may be, for example, light in 420 nm - 495 nm wavelength range that is configured to encourage photosynthesis. The light in the second wavelength range may be, for example, light in 380 nm - 420 nm wavelength range that is configured for disinfection.

The control system 403 may (e.g., using FRT, one or more sensors, or a user input, etc.) determine (e.g., identify) an item in the drawers 410. The control system 403 may, based on the determination, adjust parameters (e.g., wavelength) of light in the drawers 410. The control system 403 may activate the light emitters 402-2 (and may deactivate the light emitters 402-3), for example, if the control system 403 determines that the drawers 410 comprise items of a first type (e.g., items, such as leafy vegetables, that are susceptible to photo-degradation, etc.). The control system 403 may activate the light emitters 402-3 (and may deactivate the light emitters 402-2), for example, if the control system 403 determines that the drawers 410 do not comprise items of the first type.

Parameters (e.g., wavelengths, intensities, etc.) of disinfecting light in a first zone may be different from one or more parameters of disinfecting light in a second zone. The control system 403 may (e.g., using FRT, one or more sensors, or a user input, etc.) determine (e.g., identify) first items in the drawers 410 and second items in the shelving 406. The control system 403 may determine that the drawers 410 comprise items of a first type (e.g., items, such as leafy vegetables, that are susceptible to photo-degradation, etc.), and the shelving does not comprise items of the first type. The control system 403 may apply light in a first wavelength range (e.g., 420 nm - 495 nm) using the light emitters 402-2 (or deactivate light) in the drawers 410, and apply light in a second wavelength range (e.g., 380 nm - 420 nm) in the shelving 406 using light emitters 402-1.

A first section (e.g., the drawers 410) may be for storage of a first content type (e.g., vegetables). The control system 403 may determine (e.g., using FRT, one or more sensors, or a user input, etc.) that items in the drawers 410 are not items of the first content type, and deactivate the light emitters 402-2. A second section (e.g., the shelving 406) may be for storage of a second content type (e.g., dairy products). The control system 403 may determine (e.g., using FRT, one or more sensors, or a user input, etc.) that items in the drawers 410 are not items of the second content type but items of the first content type, and deactivate the light emitters 402-1 (e.g., to prevent photo-degradation).

The control system 403 may be configured to adjust intensities of disinfecting light to increase shelf life of certain items (e.g., meat, fruit, etc.). Higher intensities of disinfecting light may, for example, cause certain items (e.g., fruit) to dry out. The control system 403 may, for example, recognize items in a first section (e.g., shelving 406) of the appliance 400 as meat, and may reduce and/or deactivate the light emitters 402-1 in the shelving 406 to reduce degradation of the meat. The control system 403 may, for example, recognize items in the shelving 406 as fruit, and may control the light emitters 402-1 to prevent the fruit from drying out. The control system 403 may monitor one or more parameters (e.g., dosage, radiant flux, output power, irradiance, etc.) of light from the light emitters 402-1 and control the light emitters 402-1 such that the dosage is within a threshold. The control system 403 may, for example, recognize items in a second section (e.g., drawers 410) of the appliance 400 as neither meat nor fruit, and may not adjust light from the light emitters 402-3 in the second section. The control system 403 may control (e.g., based on a determined type of food) exposure time of a specific food being stored in a specific zone, a start time of disinfecting light exposure in the zone, and/or stop time disinfecting light exposure of the zone.

The control system 403 may account a “time-of-purchase” of one or more items to control parameters of disinfecting light. The “time-of-purchase” may be determined based on a user input provided via the user interface 414.

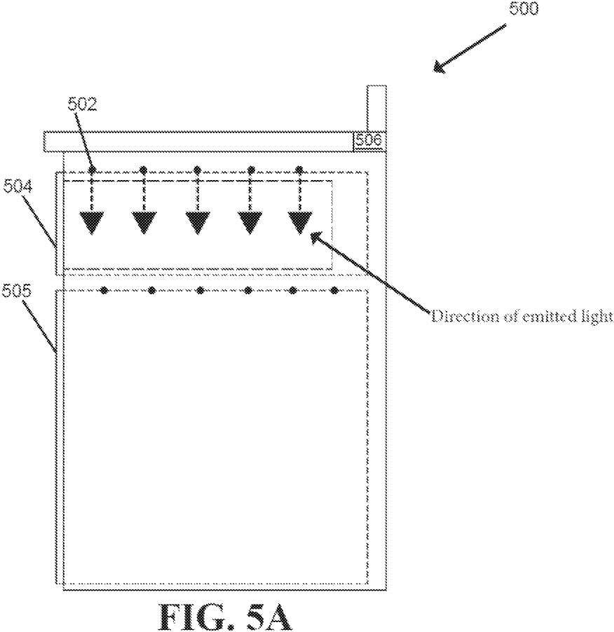

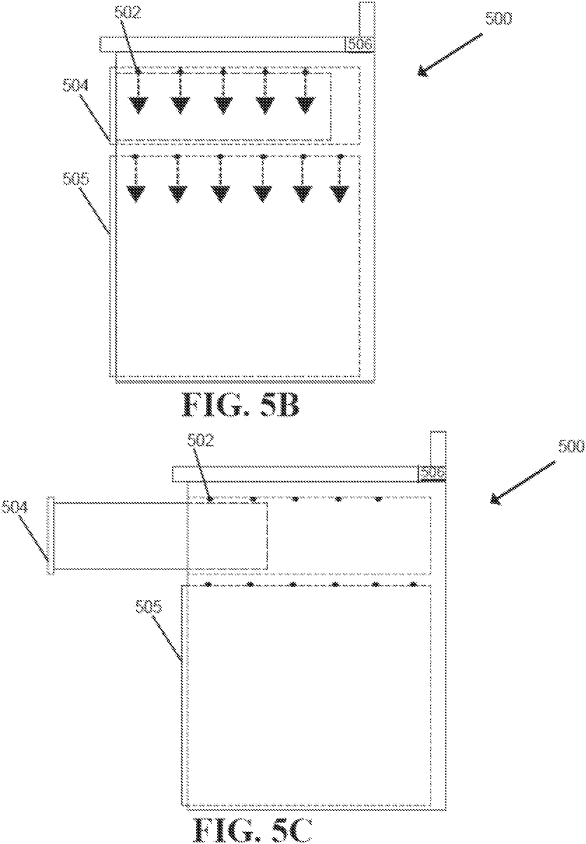

FIGS. 5A-5C show an example appliance 500 with disinfection capabilities. The appliance 500 may be a cabinet. The appliance 500 may comprise disinfecting light emitters 502, a drawer 504, and/or a base 505. The light emitters 502 may be integrated into an inside surface of the drawer 504 and/or a base 505 in a manner such that disinfecting light is directed to the interior surfaces of the appliance 500 when the drawer 504 and/or the base 505 of the appliance 500 is closed. Additionally, or alternatively, the light emitters 502 may be integrated on other locations of the appliance 500. The light emitters 502 may emit light in a 380 nm - 420 nm wavelength range (or in a portion of the wavelength range, or a specific wavelength in the wavelength range). The appliance 500 may include features described above with respect to the appliance 100, the appliance 200, the appliance 300, and/or the appliance 400.

The appliance 500 may comprise a control system 506 to control one or more parameters of disinfecting light (e.g., dosage, radiant flux, exposure time, wavelength, and/or irradiance) from the light emitters 502. The control system 506 may control the light emitters 502 based on a change of status of the appliance 500. The control system 506 may cause and/or adjust light from the light emitters 502, for example, based on whether the drawer 504 and/or the base 505 of the appliance 200 is opened/closed. For example, as shown in FIG. 5A, the control system 506 may activate the light emitters in an interior of the drawer 504 when the drawer 504 is closed. As shown in FIG. 5B, the control system 506 may activate the light emitters in both the interior of the drawer 504 and an interior of the base 505 when the drawer 504 and/or the base 505 are closed. As shown in FIG. 5C, illumination in both the interior of the drawer 504 and an interior of the base 505 may no longer be illuminated when the drawer 504 and/or the base 505 is opened. One or more sensors (e.g., motion sensors, voice sensor, light beam sensors, infrared sensors, magnetic proximity sensors, capacitive touch sensors, limit switches, irradiance sensors, intensity sensors, etc.) may be used to detect a change of status of the appliance 500. The control system 506 may detect the change in status of the appliance 500 based on measurements obtained by the one or more sensors.

One or more light emitters (e.g., the light emitters 102, 202, 302, 402, 502) may be any device capable of emitting light. Light emitters may be, for example, light emitting diodes (LEDs), LEDs with light-converting layer(s), laser, electroluminescent wires, electroluminescent sheets, flexible LEDs, and/or organic LEDs (OLEDs). The light emitters may be tunable LEDs. A control system may control output wavelengths of a tunable LED based on various considerations described herein. Light-converting materials may comprise a phosphor, an optical brightener, a combination of phosphors, a combination of optical brighteners, and/or a combination of phosphor(s) and optical brightener(s). Light-converting materials may comprise quantum dots, a phosphorescent material, a fluorophore, a fluorescent dye, a conductive polymer, or a combination of any one or more types of light-converting materials. Light-converting materials may comprise an activator (e.g., a light converting element) and a host (e.g., a non-light converting element). Light emitters may be in the form of LED strip lighting. LED strip lighting may comprise a plurality of LEDs. This configuration may allow light to hit surfaces and contents of an appliance at many different angles so the possibility of shadows is decreased. Light emitters may be point light sources (e.g., puck lights).

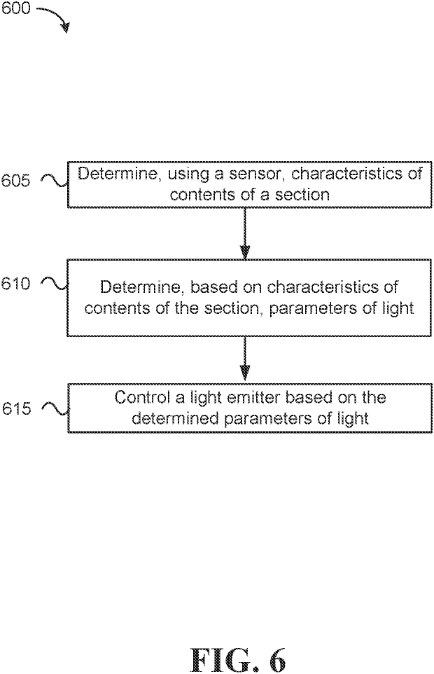

FIG. 6 shows an example method 600 for controlling light, in accordance with one or more examples described herein. A control system in an appliance described with reference to FIGS. 1-5 may implement the method 600. In other examples, a lighting device may comprise a light emitter and a controller, where the controller implements the method 600.

At step 605, a control system may determine, using a sensor, characteristics of contents in a section of the appliance. A controller, for example, may receive one or more signals, wherein the one or more signals may be generated by a sensor. The one or more signals may indicate a characteristic of a section (e.g., in an appliance). The characteristic may be, for example, weight of contents in the section, an image of contents in the section, a color of contents in the section, etc.

At step 610, the control system may determine, based on the characteristics of the contents, parameters of light. The control system may determine, for example, a dosage of light that is in proportion to a weight of contents in the section. The control system may determine a type of contents in the section, and, based on the type, may determine a dosage and/or wavelength/wavelength range of light to be used. The control system may determine color of contents in the section, and, based on the color, may determine a dosage and/or wavelength of light to be used. The parameters may be independent of contents of any other sections of the appliance and/or any other parameters of other light (e.g., used for other light emitters) in the appliance.

At step 615, the control system may control a light emitter based on the determined parameters of light. The control system may apply a dosage of light by activating the light emitter for a first duration of time and deactivating the light emitter for a second duration of time. The light emitter may be, for example, a light emitter configured to emit light in a 380 nm - 420 nm wavelength range. The light emitter may be, for example, a light emitter configured to emit light in a 420 nm - 500 nm wavelength range and the control system may activate the light emitter (e.g., for a predetermined duration of time) based on determining that the contents of the section are green in color.

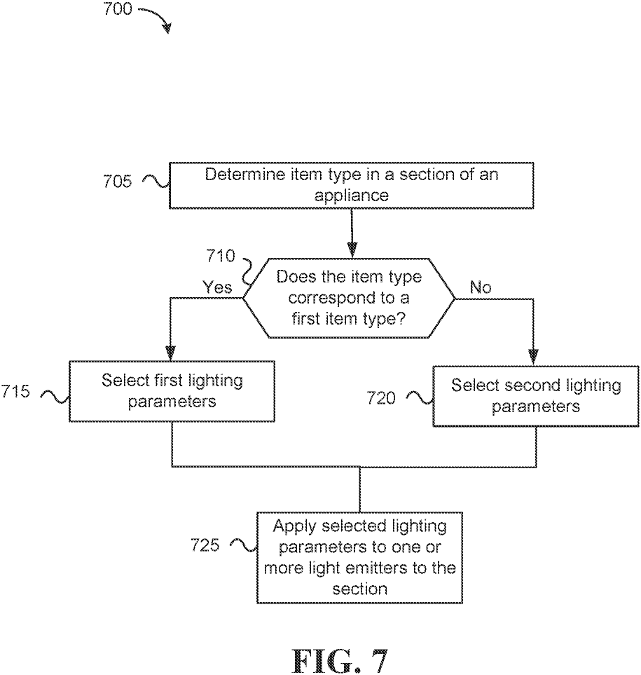

FIG. 7 shows an example method 700 for controlling light, in accordance with one or more examples described herein. A control system in an appliance described with reference to FIGS. 1-5 may implement the method 700. In other examples, a lighting device may comprise one or more light emitters, a controller, and a sensor, where the controller implements the method 700.

At step 705, the controller may determine a type of item stored in a section of the appliance. The controller may use one or more sensors to determine a type of the item. The controller may use for, example, an image sensor (e.g., camera) to detect the contents of the section. The controller may analyze an image from the image sensor to determine a color of the contents. The controller may determine that the item is a leafy green vegetable, for example, if the color is determined to be green. The controller may determine that the item is a not a leafy green vegetable, for example, if the color is determined to be a color other than green.

At step 715, the appliance may select first lighting parameters if the appliance determines that the item corresponds to a first item type. The control system may select a light wavelength range of 420 nm - 500 nm, for example, if the item is determined to be a leafy green vegetable. At step 720, the appliance may select second lighting parameters if the appliance determines that the item corresponds to a second item type. The control system may select a light wavelength range of 380 nm - 420 nm, for example, if the item is determined not to be a leafy green vegetable. Other parameters that may be controlled may comprise output power of the applied light, dosages of applied light, irradiance of applied light, etc.

At step 725, the controller may apply the selected lighting parameters. The controller may, for example, activate a first light emitter configured to emit light in a wavelength range of 420 nm - 500 nm, for example, if the controller selects the first lighting parameters. The controller may, for example, activate a second light emitter configured to emit light in a wavelength range of 380 nm - 420 nm, for example, if the controller selects the second lighting parameters. The controller may deactivate the first light emitter or the second light emitter after a particular (e.g., a predetermined) dosage is achieved.

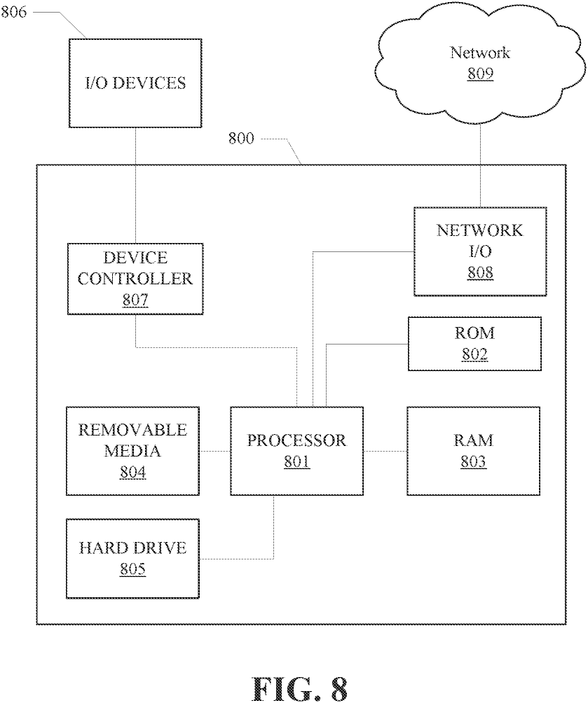

FIG. 8 illustrates an example computing device 800 (e.g., a controller), that may perform the methods 600 and/or 700, the functions of various control systems (e.g., control systems 106, 206, 403, 506) described herein, and/or any other computer, controller, or processor-based function described herein. The computing device 800 may implement, for example, a control system for control of various lighting parameters, as described herein. In some examples, the computing device 800, in communication with one or more sensors and one or more lighting devices may implement lighting controls based on sensor measurements. In some examples, the computing device 800 may be a microcontroller configured to implement the functions of various control systems described herein.

The computing device 800 may include one or more processors 801, which may execute instructions of a computer program to perform any of the features described herein. The instructions may be stored in any type of tangible computer-readable medium or memory, to configure the operation of the processor 801. As used herein, the term tangible computer-readable storage medium is expressly defined to include storage devices or storage discs and to exclude transmission media and propagating signals. For example, instructions may be stored in a read-only memory (ROM) 802, random access memory (RAM) 803, or removable media 804, such as a Universal Serial Bus (USB) drive, compact disc (CD) or digital versatile disc (DVD), floppy disk drive, or any other desired electronic storage medium. Instructions may also be stored in an attached (or internal) hard drive 805. The computing device 800 may include one or more input/output devices 806, such as one or more sensors, lighting devices, display, touch screen, keyboard, mouse, microphone, software user interface, etc. The computing device 800 may include one or more device controllers 807 such as a video processor, keyboard controller, etc. The computing device 800 may also include one or more network interfaces 808, such as input/output circuits (such as a network card) to communicate with a network such as example network 809. The network interface 808 may be a wired interface, wireless interface, or a combination thereof. The computing device 800 may comprise one or more timers to measure time. One or more of the elements described above may be removed, rearranged, or supplemented without departing from the scope of the present disclosure.

Various methods, devices, and systems described herein may use a control system (e.g., a control system described with reference to FIG. 8) to implement various lighting controls in appliances (e.g., the appliances 100, 200, 300, 400, 500). The control system may be used to control/adjust various aspects of disinfecting light (e.g., dosage, radiant flux, color, time, wavelength, intensity, and/or irradiance). In various examples, the control system may be used to control similar parameters corresponding to other wavelengths of light as well. The other wavelengths of light may correspond to white light, ultraviolet (UV) light, and/or other wavelengths that are not configured for disinfection.

The input/output devices 806 may comprise light source(s) configured to provide light disinfecting light (e.g., 380 nm - 420 nm wavelength range), and/or other wavelengths of light. The input/output devices 806 may comprise sensor(s). The sensor(s) may be used to determine one or more parameters corresponding to an environment subject to inactivating light and/or disinfecting light. The sensor(s) may sense any parameter of a control environment of an appliance, including but not limited to: touch of the appliance, heat of a user’s hand on the device, motion of a user, motion of structure to which the appliance may be coupled, temperature, light reception, and/or presence of microorganisms on exterior surface, etc. The sensor(s) may include any now-known or later-developed sensing devices for the desired parameter(s). The sensor(s) may comprise, for example, one or more of irradiance sensors, radiant intensity sensors, motion sensors, voice sensors, odor sensors, capacitive touch sensors, magnetic proximity sensors, light sensors, infrared sensors, cameras, ultrasonic sensors, weight sensors, limit switches, and/or any other sensors. The sensor(s) may send the detected information to the computing device 800. The computing device 800 may control an output of the light source(s) based on one or more measurements determined using the one or more sensors.

The appliances, the light emitters, and/or the control systems disclosed herein may be powered through power outlets, electrical power supplies, batteries or rechargeable batteries mounted in proximity to the appliance, and/or wireless or inductive charging. An appliance, along with associated light emitters and control system may be powered through a single electrical outlet. Rechargeable batteries, if used in the appliances, may be recharged, for example, using AC power or solar panels (e.g., if sufficient sunlight is available).

An example appliance may comprise a first section, a second section, a first light emitter configured to emit a first light, having a first peak wavelength in a first wavelength range of 380 nm - 420 nm, into the first section, and a control system configured to control, based on a characteristic of the first section, a first characteristic of the first light in the first section. The control system may be further configured to control a second characteristic of a second light in the second section. The first characteristic of the first light in the first section may be independent of the second characteristic of the second light in the second section.

The appliance may further comprise a sensor in communication with the control system and configured to determine the characteristic of the first section. The control system may be configured to adjust the first characteristic of the first light based on the determined characteristic of the first section. The determined characteristic of the first section may be an occupancy of the first section. The control system may adjust the first light based on the occupancy of the first section. The first light emitter may be configured to emit the first light at a first time, and may be further configured to emit the second light at a second time. The second section may comprise the first section. The second light configured in the second section may have a second peak wavelength in: the first wavelength range of 380 nm - 420 nm, or a second wavelength range of 420 nm - 495 nm. The first light emitter may be configured to emit the first light, through the second section into the first section. The first characteristic of the first light comprise at least one of: a radiant flux of the first light emitter, a dosage of the first light, or a wavelength range of the first light. The second section may comprise a light filtering material that is configured to block light in the first wavelength range.

An example appliance may comprise a first section, a second section, a first light emitter configured to emit first light having a first peak wavelength in a first wavelength range of 380 nm - 420 nm into the first section, a second light emitter configured to emit second light into the second section, and a sensor. An example method of controlling light in an interior of the appliance may comprise receiving, from the sensor, sensor data, where the sensor data may comprise an indication of a characteristic of contents of the first section. The example method may further comprise determining, based on the characteristic of contents of the first section, a dosage of the first light to be provided to the first section over a period of time. The example method may further comprise emitting, using the first light emitter, a first radiant flux of the first light over the period of time to provide the determined dosage, where the first radiant flux of the first light is independent of a second radiant flux of the second light.

The second light configured in the second section may have a second peak wavelength in: the first wavelength range of 380 nm - 420 nm, or a second wavelength range of 420 nm -495 nm. The example method may further comprise determining, based on a determination that the first section is occupied at a first time, to emit the first radiant flux at a first level at the first time. The example method may further comprise determining, based on a determination that the first section is unoccupied at a second time, to emit the first radiant flux at a second level lower than the first level at the second time. The characteristic may be a level of occupancy in the first section. The dosage of the first light may be proportional to the level of occupancy in the first section. The example method may further comprise determining, based on a determination that the first section comprises a first item type at a first time, to activate the first light emitter at the first time. The example method may further comprise determining, based on a determination that the first section comprises a second item type at a second time, to deactivate the first light emitter at the second time. The characteristic of the contents of the first section is at least one of: an outline of an image of the contents in the first section; a color of the contents in the first section.

An example device may comprise one or more light emitters, a sensor configured to determine an occupancy in the first section of the appliance and a control system. The one or more light emitters may be configured to emit a first light, having a first peak wavelength in a first wavelength range of 380 nm - 420 nm, into a section of an appliance. The one or more light emitters may be configured to emit a second light, having a peak wavelength in a second wavelength range of 420 nm - 495 nm, into the section of the appliance. The control system may be configured to control a first radiant flux of the first light and a second radiant flux of the second light in at least the section of the appliance based on the determined occupancy.

The sensor may comprise a camera, a motion sensor, or a weight sensor. The control system may be further configured to determine a type of contents in the section, and adjust the first radiant flux based on the determined type of the contents. The control system may be further configured to determine a motion of contents in the section, and adjust the first radiant flux in proportion to a frequency of the motion of the contents in the section. The control system may be further configured to determine, based on a determination that the section is occupied at a first time, to emit the first radiant flux at a first level at the first time. The control system may be further configured to determine, based on a determination that the section is unoccupied at a second time, to emit the first radiant flux at a second level higher than the first level at the second time. A single tunable LED may emit the first light at a first time and the second light at a second time.

The above discussed embodiments are simply examples, and modifications may be made as desired for different implementations. For example, steps and/or components may be subdivided, combined, rearranged, removed, and/or augmented; performed on a single device or a plurality of devices; performed in parallel, in series; or any combination thereof. Additional features may be added.

1. An appliance comprising a section and a light emitter configured to emit light, having a peak wavelength in a wavelength range of 380 nm - 420 nm, into the section.
